(19) United States
(12) Patent Application Publication
(10) Pub. No.: US 2012/0265091 A1
(43) Pub. Date: Oct. 18, 2012

(54) Device and method for monitoring the success of spinal anesthesia

(75) Inventors: Andreas Penno, Lubeck (DE); Ulf Grossmann, Schoenberg/Kalifomien (DE)

(73) Assignee: Andreas Penno, Lübeck (DE)

(21) Appl. No.: 13/142,762

(22) PCT Filed: Dec. 23, 2009
(86) PCT No.: PCT/EP2009/009238

§ 371 Date: Apr. 19, 2012

(30) Foreign Application Priority Data
Jan. 3, 2009 (DE) 10 2009 003 897.3

(51) Int. Cl. A61B 5/01

(52) U.S. Cl. 600/549

(57) ABSTRACT

A device for the monitoring of the success of spinal anesthesia with
at least one electronic temperature sensor for measuring the skin surface temperature within at least one dermatome of a patient,
an electronic evaluation device, which is connected with the at least one temperature sensor and is designed such that it monitors the measurement signal delivered by the at least one temperature sensor to determine whether the skin surface temperature increases by approximately 2 to 3° C., and
a display device that displays the result of the evaluation by the electronic evaluation device optically and/or acoustically.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

The object of the invention is a device and a method for monitoring the success of spinal anesthesia in medicine.

Spinal anesthesia is a form of regional anesthesia close to the spinal cord. Through the injection of a local anesthesia into the cerebrospinal fluid area at the height of the lumbar spine, the signal transfer in the nerves extending from the spinal cord is inhibited. A temporary, reversible blockage of the sympathetic nervous system, the sensitivity and the motor function of the lower half of the body is thereby achieved.

As a standard procedure in anesthesia, spinal anesthesia is used today in a plurality of operations in the lower stomach, the pelvis, the lower extremities and in obstetrics and represents an alternative to other regional procedures such as peridural anesthesia and full narcosis.

The human spinal column consists of 34 vertebrae, which are connected by firm bands and surround the spinal cord. Spinal nerves extend out between the vertebrae, which innervate the body segmentally and enable sensitivity and also carry fibers of the vegetative nervous system (sympathetic/parasympathetic nervous systems). As part of the central nervous system, the spinal cord is surrounded by the meninges, which restrict the cerebrospinal fluid area, in which the cerebrospinal fluid circulates. This cerebrospinal fluid space is punctured with a thin cannula during spinal anesthesia. Local anesthesia is injected through the tip of the needle, which acts on the front and rear roots of the spinal nerves and temporarily blocks their ability to transmit nerve impulses.

It is known that, in spinal anesthesia, different qualities of the sensor system can be switched off in succession: first, the preganglionic sympathetic nervous system of the vegetative nervous system is blocked. This results in vessel dilation, a warming of the skin and an eventual drop in blood pressure. Pain and temperature fibers are then blocked. Touch and pressure sensation follows. Motor activity and sense of vibration and location is switched off last.

The effective height of the spinal anesthesia depends on the dispersion of the injected activate agents in the cerebrospinal fluid, which can be influenced through dose and concentration of the local anesthesia through the positioning of the patient.

So far, there is no device that can show the anesthetization for this form of anesthesia. The analgesia has previously been tested by means of a cold spray by the anesthetist on the body of the patient. The patient needed to verbally indicate whether or not he/she still felt a corresponding cold sensation.

Based on this, the object of the invention is to provide a technique for facilitating the monitoring of the success of spinal anesthesia.

### BRIEF SUMMARY OF THE INVENTION

The device according to the invention for monitoring the success of spinal anesthesia has
at least one electronic temperature sensor for measuring the skin surface temperature within at least one dermatome of a patient,
an electronic evaluation device, which is connected with the at least one temperature sensor and is designed such that it monitors the measurement signal delivered by the at least one temperature sensor to determine whether the skin surface temperature increases by approximately 2 to 3° C.,
wherein the electronic evaluation device is also designed such that in the case of the determination of an increase in the skin surface temperature by approx. 2 to 3° C. it determines that the analgesia has occurred in a dermatome, which is closer to the sacral lumbar vertebrae by approximately 2 to 6 dermatomes than the dermatome, in which the temperature sensor (2) measures the skin surface temperature, and
a display device that displays the result of the evaluation by the electronic evaluation device optically and/or acoustically.

The device according to the invention assumes that, in the case of the sympatholysis (exclusion of the sympathetic innervation) by the spinal anesthesia, the “thinner” non-myelinated nerve fibers are blocked first and only then the “thicker” myelinated nerve fibers. As a result, during the spinal anesthesia, the blockage of the sympathetic nervous system is generally two to three segments or respectively dermatomes further away than the sensory blockage. The blockage of the sympathetic nervous system can be up to six segments or respectively dermatomes further away than the sensory blockage. Furthermore, the sensory blockage is approx. two segments or respectively dermatomes further away than the motor blockage. The blockage of the sympathetic nervous system accompanies an increase in the skin temperature by approximately 2 to 3° C. in the associated dermatome. This temperature increase thus indicates that a sensory blockage and thus the analgesia (anesthetization) has occurred approximately 2 to 3 (up to 6) dermatomes more caudally. For example, in the case of an increase in the temperature of the skin surface by approximately 2 to 3° C. in dermatome Th2, the analgesia should be assumed approximately 2 to 3 (−6) dermatomes more caudally in dermatomes Th4 through Th5 (−Th8). The invention takes advantage of this knowledge in that it makes it possible, due to the measured temperature increase of approximately 2 to 3° C. at at least one dermatome of a patient, to identify the dermatome arranged 2 to 3 (−6 dermatomes) more caudally than the one in which the analgesia already occurred. For example, through display of the dermatome, in which the temperature increase of 2 to 3° C. occurred, and if applicable the dermatomes, in which this temperature increase already occurred previously, the checking of the success of the spinal anesthesia will be easier for the healthcare professional. The previous check by means of cold spray and verbal reaction of the patient is replaced by an objective measurement and evaluation of the measurement results.

A simple design of the device according to the invention shows as a result of the evaluation by the evaluation device that or respectively those dermatomes, in which the temperature increase of approximately 2 to 3° C. occurred. Based on this display, it can be assumed that the analgesia has occurred approximately 2 to 3 (up to 6) dermatomes more caudally. The electronic evaluation device determines that the analgesia has occurred in a dermatome, which is closer to the sacral lumbar vertebrae by approximately 2 to 6 dermatomes than the dermatome, in which a temperature increase of the skin surface of approximately 2 to 3° C. was determined. The display device shows the dermatome, in which the analgesia occurred, as a result of the evaluation by the electronic evaluation device. If applicable, the display device also shows the dermatomes, in which the analgesia already occurred previously. The electronic evaluation device preferably determines that the analgesia has occurred in a dermatome, which is closer to the sacral lumbar vertebrae by approximately 2 to 3 dermatomes than the dermatome, in which a temperature increase of the skin surface of approximately 2 to 3° C. was determined. In accordance with another embodiment, the display device shows the dermatomes, in which the skin surface temperature is increased by 2 to 3° C., and the dermatomes, in which the analgesia has occurred (e.g. in different colors).

The temperature sensor can be designed differently. For example, it is possible to measure the skin surface temperature of the patient over a large area with a thermal camera and to determine the temperatures in the individual dermatomes with an automatic image evaluation procedure. In accordance with another embodiment, individual temperature sensors are used. In accordance with a preferred embodiment, the at least one temperature sensor is an NTC resistor.

In accordance with one embodiment of the invention, at least one temperature sensor is held via spring means on means for fastening the temperature sensor on the body of a patient. The temperature sensor is pressed against the skin surface with a constant pressing force via spring means. Measurement errors are hereby avoided.

As a general rule, the temperature sensors can be secured individually to the skin surface of the patient. In accordance with one embodiment, several temperature sensors are arranged at certain distances from each other on a band, which can be fastened by means for fastening on the body of a patient. Through the specified arrangement of the temperature sensors on a band, the placement of the temperature sensors on the body of the patient is facilitated. The distance between two neighboring temperature sensors on the band is preferably the distance of one or more dermatomes. This embodiment facilitates the attachment of the temperature sensors within the different dermatomes of the patient. The measurement of the distance of neighboring temperature sensors on the band can be based on an average-sized patient. Furthermore, it is possible to provide several bands with temperature sensors at different distances for patients of a different size.

In accordance with one embodiment, the temperature sensor is connected with a tape for fastening on the body of a patient.

The electronic evaluation device can be analog or digital. It can be a program-controlled electronic data-processing device or pure hardware. A programmable, digital evaluation device is preferably used. In particular, a PC can be used as the evaluation device.

In accordance with one embodiment, an analog temperature sensor is connected with a digital evaluation device via at least one analog-digital converter. In accordance with another embodiment, the analog-digital converter is connected to a USB port of the PC.

The display device is for example a monitor of a PC. In accordance with one embodiment, the display device shows a graphic of a human body, in which the dermatomes, in which the analgesia has occurred, and/or the dermatomes, in which the skin surface temperature has increased by approximately 2 to 3° C., are highlighted graphically. The graphical highlighting can take place e.g. by coloring the concerned dermatomes a different color than the rest of the graphic.

### BRIEF DESCRIPTION OF THE SEVERAL VIEWS OF THE DRAWINGS

The invention is explained in greater detail below based on the attached drawings of an exemplary embodiment. The drawings show the following:

FIG. 1 a device according to the invention for monitoring the success of spinal anesthesia on a body of a patient in a rough, schematic block diagram;

FIG. 2 a section of a band with temperature sensors in a top view;

FIG. 3 an enlarged detail of the band from FIG. 2 in a vertical cut.

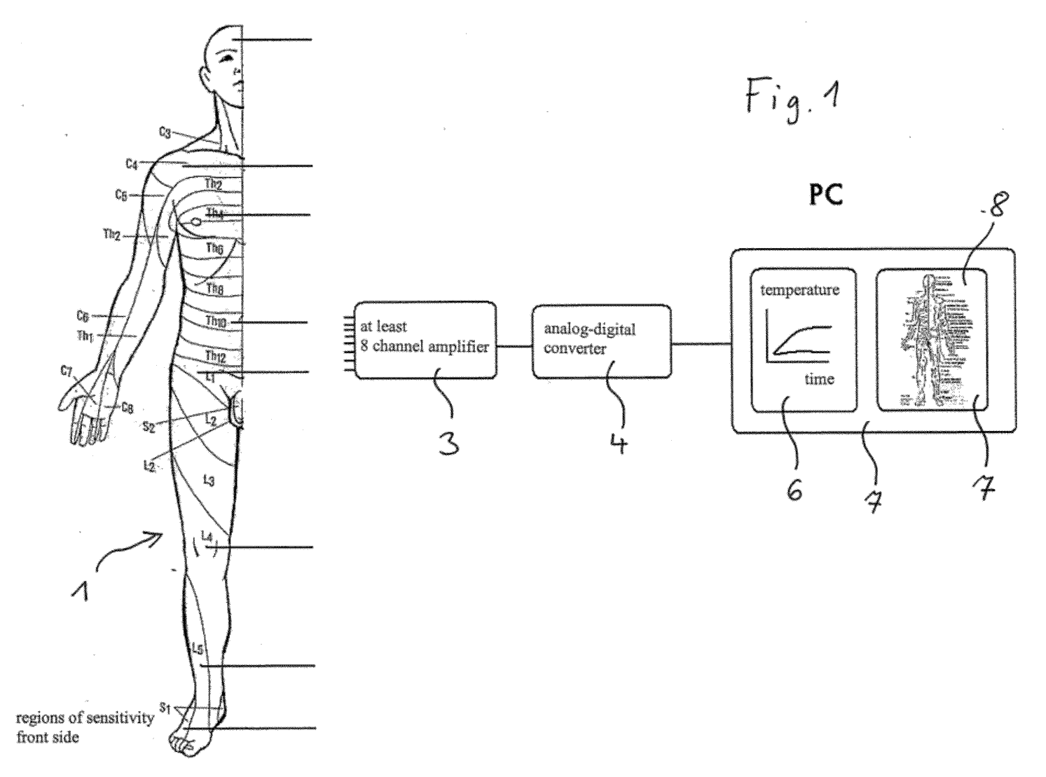

In accordance with FIG. 1, the skin surface of a patient 1 is segmented into different dermatomes, which are labeled with reference symbols such as Th2, L3 and C4. Temperature sensors 2 are attached to certain dermatomes.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated

In accordance with FIG. 1, the skin surface of a patient 1 is segmented into different dermatomes, which are labeled with reference symbols such as Th2, L3 and C4. Temperature sensors 2 are attached to certain dermatomes. The temperature sensors 2 are connected with an analog-digital converter 4 via an amplifier 3 with at least 8 channels. The analog-digital converter 4 scans the output channels of the amplifier 3 and converts the amplified, analog measurement signal into a digital signal.

The analog-digital converter 4 is attached to a PC 5. The PC 5 determines whether the skin temperature measured by the temperature sensors 2 increases by 2 to 3° C. as shown in the temperature-time diagram 6. If the PC 5 determines an increase by 2 to 3° C., it calculates that an analgesia has occurred in a dermatome arranged approximately 2 to 3 (−6) dermatomes more caudally. The dermatomes, in which the analgesia was determined, are displayed on a screen 7 with a graphic 8 of a human body.

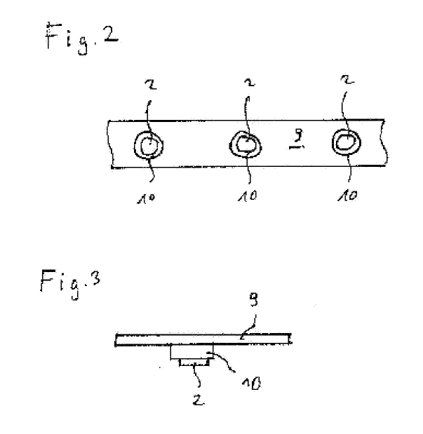

In accordance with FIGS. 2 and 3, several temperature sensors 2 are fastened on a band 9 each under an intermediate layer of a foam cushion. The distances between neighboring temperature sensors 2 correspond with the distances between certain dermatomes. The band 9 is fastenable on the body 1 with tapes which are attached transversely on the band 9.

This completes the description of the preferred and alternate embodiments of the invention. Those skilled in the art may recognize other equivalents to the specific embodiment described herein which equivalents are intended to be encompassed by the claims attached hereto.

1-22. (canceled)

23. A device for the monitoring of the success of spinal anesthesia with
at least one electronic temperature sensor (2) for measuring the skin surface temperature within at least one dermatome of a patient, an electronic evaluation device (5), which is connected with the at least one temperature sensor (2) and is designed such that it monitors the measurement signal delivered by the at least one temperature sensor (2) to determine whether the skin surface temperature increases by approximately 2 to 3° C.,
wherein the electronic evaluation device (5) is also designed such that in the case of the determination of an increase in the skin surface temperature by approx. 2 to 3° C. it determines that the analgesia has occurred in a dermatome, which is closer to the sacral lumbar vertebrae by approximately 2 to 6 dermatomes than the dermatome, in which the temperature sensor (2) measures the skin surface temperature, and
a display device (7) that displays the result of the evaluation by the electronic evaluation device optically and/or acoustically.

24. The device according to claim 23, in which the electronic evaluation unit (5) is designed such that in the case of the determination of an increase in the skin surface temperature by approx. 2 to 3° C. it determines that the analgesia has occurred in a dermatome, which is closer to the sacral lumbar vertebrae by approximately 2 to 3 dermatomes than the dermatome, in which the temperature sensor (2) measures the skin surface temperature.

25. The device according to claim 23, in which the at least one temperature sensor (2) is an NTC resistor.

26. The device according to claim 23, in which at least one temperature sensor (2) is held via spring means on means for fastening (9) on the body of a patient.

27. The device according to claim 23, in which several temperature sensors (2) are arranged on a band (9) at certain distances from each other, which is fastenable with means for fastening on the body of a patient.

28. The device according to claim 23, in which the at least one temperature sensor is connected with a tape for fastening on the body of a patient.

29. The device according to claim 27, in which the distances between two neighboring temperature sensors (2) on the band (9) corresponds with the distances between one or more dermatomes.

30. The device according to claim 23, in which the at least one temperature sensor (2) is connected with a digital evaluation device (5) via at least one analog-digital converter (4).

31. The device according to claim 30, in which the digital evaluation device (5) is a PC.

32. The device according to claim 30, in which the analog-digital converter (4) is connected with a USB port of the PC (5).

33. The device according to claim 23, in which the display device (7) shows a graphic (8) of a human body, in which the dermatomes, in which the analgesia have occurred, and/or the dermatomes, in which the skin surface temperature has increased by approximately 2 to 3° C., are highlighted graphically.

34. The method for monitoring the success of spinal anesthesia, in which at least one electronic temperature sensor measures the skin surface temperature within at least one dermatome of a patient,
an electronic evaluation device monitors the measurement signal delivered by the at least one temperature sensor for an increase in the skin surface temperature by approximately 2 to 3° C.,
the electronic evaluation device in the case of an increase in the skin surface temperature by approximately 2 to 3° C. determines the analgesia in a dermatome closer to the sacral lumbar vertebrae of the patient by 2 to 6 dermatomes than the temperature sensor and
a display device shows the result of the evaluation by the electronic evaluation device.

35. The method according to claim 34, in which the display device display the analgesia in the determined dermatome.

36. The method according to claim 34, in which the electronic evaluation device determines, in the case of an increase in the skin surface temperature by approximately 2 to 3° C., the analgesia in a dermatome closer to the sacral lumbar vertebrae of the patient by 2 to 3 dermatomes than the temperature sensor and the display device displays the analgesia in the determined dermatome.

37. The method according to claim 34, in which at least one temperature sensor measures the skin surface within different dermatomes.

38. The method according to claim 37, in which at least one temperature sensor is arranged on the skin surface.

39. The method according to claim 38, in which several temperature sensors are arranged on the skin surface of the patient at a distance of at least one dermatome.

40. The method according to claim 38, in which at least one sensor is pressed against the skin surface via a spring means.

41. The method according to claim 38, in which the temperature sensors are placed on the skin surface of the patient by means of a band, on which they are arranged at certain distances from each other.

42. The method according to claim 34, in which the temperature sensors deliver an analog measurement signal, which is converted into a digital signal by means of the analog-digital converter and which is digitally processed by the evaluation device.

43. The method according to claim 34, in which the display device displays in a graphically highlighted manner in a graphic of a human body the dermatomes, in which the analgesia has occurred, and/or the dermatomes, in which the skin surface has increased by approximately 2 to 3° C.

44. The method according to claim 34, in which the evaluation device displays the result of the evaluation optically and/or acoustically.
